(19) United States
(12) Patent Application Publication
(10) Pub. No.: US 2005/0226894 A1
(43) Pub. Date: Oct. 13, 2005

(54) Oral vaccination

(76) Inventors: Jensen-Jarolim Erika, Vienna (AT); Scheiner Otto, Perchtoldsdorf (AT); Pehamberger Hubert, Vienna (AT); Zielinski Christoph, Vienna (AT); Breiteneder Heimo, Vienna (AT)

Correspondence Address: MILBANK, TWEED, HADLEY & MCCLOY LLP, INTERNATIONAL SQUARE BUILDING, 1825 EYE STRET, N.W. #1100, WASHINGTON, DC, 20006, US

(21) Appl. No.: 10/501,402

(22) PCT Filed: Jan. 15, 2003
(86) PCT No.: PCT/EP03/00369

§ 371 Date: Jan. 3, 2005

(30) Foreign Application Priority Data
Jan. 15, 2002 (EP) 02000901.5
Dec. 17, 2002 (DE) 102 59 053.2

(51) Int. Cl. A61K 39/02; A61K 33/06; A61K 31/4439

(52) U.S. Cl. 424/234.1; 514/338; 424/682

(57) ABSTRACT

The invention relates to a drug for oral vaccination, the drug containing an antigenically active substance and a gastric acid reducing substance as a combination preparation for joint or separate, simultaneous or time shifted, oral application for vaccination.

The present invention relates to a drug for oral vaccination.

It is frequently desirable in medicine to fight diseases not only symptomatically but to prevent them from arising in the first place. The vaccination method has therefore been adopted in the area of infectious diseases but recently also in the area of cancerous diseases. This method enables the body to react to pathogenic substances, also called antigens, with the help of its immune system. This method, also known as active immunization, works by presenting an antigen associated with a disease to the immune system, whereupon the immune system reacts by forming corresponding specific antibodies against said antigens. Said so-called antigens frequently consist of peptides or proteins, that is, amino acid sequences, which are frequently administered parenterally, i.e. so as to avoid the gastrointestinal tract. This is usually done by means of injection or infusion, which is frequently felt to be unpleasant by patients.

It is accordingly the problem of the present invention to provide a vaccine which makes it possible to avoid vaccination by the parenteral route and thereby increase acceptance among patients.

The invention is based on the finding that parenteral application can be avoided if suitable conditions prevail in the gastric region which do not impair the effectiveness of the vaccine when taken up through the gastrointestinal tract and thus permit the formation of antibodies.

The subject matter of the present invention comprises drugs containing an antigenically active substance and a gastric acid reducing substance as a combination preparation for joint or separate, simultaneous or time shifted, oral application for vaccination.

Said drugs firstly make it possible for the antigenically active substance to be taken simultaneously with the gastric acid reducing substance, whereby the two phases can be present together or separately. Secondly, the combination preparation can also be applied in such a way that the antigenically active substance is administered separately and after the application of a suitable dose of gastric acid reducing substance. It is important here that gastric acid is reduced in comparison with “normal gastric acid content” at the time of application, that is, at the time when the vaccine reaches the stomach. This makes it possible for a vaccine traditionally administered parenterally to be applied orally and produce its action in the form of the generation of an immune response. In particular, the immune response generated involves immunoglobulins of groups IgE and IgG1. Furthermore, the reduced acidity also results in nonactivation of gastric proteases, since pepsinogens are only split into active pepsins by low pH in the stomach.

Since animals for example can both develop tumors and are able to produce IgE, a vaccination in accordance with the present invention might be considered in the veterinary field for example. Further, it is also suspected that IgE plays a part in warding off parasitical infections in animals, which also results in an application area of the present inventive vaccination in this field. The inventive drugs are therefore preferably used for vaccination of animals, in particular mammals. The inventive drugs are used particularly preferably for vaccination of humans.

The term “antigenically active substance” is to be understood according to the present invention as any kind of substance that causes an immune response when the substance is taken up by the body.

Examples of such substances are cancer antigens, antigens from the groups of infectious diseases, and allergy antigens (allergens), such as food allergens, as well as mimotopes of such antigens.

The antigenically active substance preferably involves so-called cancer or tumor antigens or the mimotopes thereof. These are substances of natural or synthetic origin which generate an immune response against cancer or tumors. Epitopes of naturally occurring tumor antigens are also to be understood by the term “tumor antigen” here. The cancer or tumor antigens or mimotopes thereof are preferably peptides. For example, a mimotope of the HER2/neu antigen, which occurs in different kinds of cancer such as breast cancer, is a peptide with the amino acid sequence Gln Met Trp Ala Pro Gin Trp Gly Pro Asp (sequence number 1).

The cancer or tumor antigens or mimotopes thereof can, but need not be, coupled to a carrier.

When drugs comprising an antigenically active substance and a gastric acid reducing substance are administered simultaneously, the antigenically active substance can be released in the stomach simultaneously or with a time delay in comparison with the gastric acid reducing substance. It is only important here that the gastric environment is adjusted so that the antigenically active substance can produce its action in terms of the generation of an immune response. Some individuals can have reduced gastric acid for different reasons, induced pathophysiolgically or iatrogenically. For the inventive vaccination, however, it is necessary to influence the acid content in specific and controlled fashion. In particular, it is suitable to adjust the pH of the stomach to a pH in the range of 4 to 7, preferably 4.3 to 6.5, particularly preferably 4.8 to 6. Pepsinogens are not converted to active protease in said pH ranges.

The antigen therefore remains conformationally intact under hypoacid conditions, whereby conformation epitopes are possibly decisive for the induction of IgE antibodies. An elevated resorption rate of the antigen through the stomach wall could hitherto not be ascertained but is not ruled out.

The gastric acid reducing substance preferably consists of a substance inhibiting gastric acid formation and/or binding gastric acid. It is thus ensured that the manner in which gastric acid is reduced can be based on different mechanisms of action. Such agents permit the gastric acid concentration to be adjusted to the needs of the inventive vaccination.

The gastric acid reducing agents are preferably selected from the families of active agents of antacids, H₂ receptor antagonists or proton pump inhibitors. The administration of antacids, i.e. alkaline agents, reduces the gastric acid present. H₂ receptor antagonists, however, inhibit histamine H₂ receptors competitively and reversively, thereby reducing gastric acid formation. Proton pump inhibitors for their part reduce gastric acid by direct influencing the acid secretion.

Some examples of antacids are the following active agents: sodium hydrogencarbonate, calcium carbonate, magnesium carbonate, magnesium hydroxide and magnesium hydroxide gel, magnesium silicate, aluminum phosphate, aluminum hydroxide and aluminum hydroxide gel, hydrotalcites, magaldrates, dihydroaluminum sodium carbonate, magnesium aluminate hydrate, aminoacetic acid and bismuth salts. Some substances acting protectively through the mucous membrane are carbenoxolone and sucralfate (aluminum hydroxide and sucrose sulfate). Some examples of H₂ receptor blockers are cimetidine, ranitidine, oxmetidine, famotidine, roxatidine and nizatidine. Some examples of proton pump inhibitors are omeprazole, lansoprazole, pantoprazole and rabeprazole. Since anticholinergics cause, among other things, an inhibition of salivary and gastric juice secretion, representatives of this class also can be used, e.g. pirenzepine.

The gastric acid reducing agents are particularly preferably selected from the agents, ranitidine hydrochloride and aluminum sucrose hydrogen sulfate. Both agents are suitable for the inventive drugs and generally well tolerated.

The antigenically active substances used are preferably natural or synthetic antigens and/or antigen mimotopes. As mentioned above, cancer or tumor antigens or the mimotopes thereof are preferred. These are particularly preferably peptides which can be coupled to a carrier. The natural or synthetic antigens or antigen mimotopes or mixtures thereof used cause an immune response relating to the formation of immunoglobulins after oral intake in connection with the gastric acid reducing substance. The immunoglobulins IgE and IgG1 are preferably formed.

In a preferred embodiment, the antigen or mimotope thereof is conjugated with a carrier.

Suitable carriers are molecules to which one or more antigens or mimotopes thereof can be bound chemically.

The antigen or mimotope is preferably conjugated with an immunogenic carrier.

Such carriers can be macromolecules of any kind which are able to have an immunostimulating effect. However, it is of importance that a selected carrier is well tolerated by animals and in particular by humans, i.e. is nontoxic and involves no dangers of e.g. a phage or phage particle with respect to any contained toxins or the possibility of infection e.g. of intestinal bacteria, and is nonpoisonous and triggers no serum sicknesses or food allergies. Conjugation with a carrier has the consequence, of increasing the immunogenicity of the vaccine. Some examples of carriers are keyhole limpet hemocyanin (KLH), tetanus toxoid (TT), albumen binding protein (ABP) or bovine serum albumen (BSA). The peptide or its functional variant is preferably conjugated to keyhole limpet hemocyanin (KLH) or tetanus toxoid (TT).

In a further preferred embodiment, the carrier consists of a multimer of the amino acid, lysine. This polylysine is preferably constructed as a dendrimer, i.e. as a polymer with several branches, which provides several functional groups, in particular amino groups, so that several peptides can be bound to the carrier. This purely synthetic variant is referred to as “multiple antigenic peptide” (MAP) and likewise leads to an increase of immunogenicity without using an immunogenic carrier. This can be found in the publications by Tam J P, PNAS 1988; 85: 5409-13: “Synthetic peptide vaccine design: synthesis and properties of a high density multiple antigenic peptide,” and by Olszewska W. et al. Virology 2000; 20: 98-105: “Protection against measles virus-induced encephalitis by anti-mimotope antibodies: the role of antibody affinity.”

Conjugation of the peptides to the supporting material can be effected in any way, for example by genetic engineering or chemical means, this is, the carrier and a functional group on the peptide are joined by a chemical reaction. The joining is preferably found at one end of the peptide. By genetic engineering the coupling of the protein carrier molecule with the peptide or its variant can be produced by inserting a DNA or RNA sequence coding for the total sequence of the conjugate into an expression system from which the total conjugate is then expressed. This form of conjugation can of course only be used if the total conjugate is also a protein molecule.

In a further preferred embodiment, the peptide is conjugated with a carrier via a linker. Said linker firstly serves as a spacer for the carrier but also improves coupling thereto. The linker is preferably joined to the peptide sequence or synthesized therewith.

A suitable linker is for example pentameric glycine which is joined to the C-terminus of the peptide. To facilitate the coupling reaction to a carrier protein, a C-terminal cysteine can be inserted. The peptide can thus be coupled to the carrier with a linker via a disulfide bridge. A further suitable linker is the motif GPGPG which can likewise be extended by a cysteine residue to the motif GPGPGC to obtain a disulfide bridge bond to the carrier protein.

It is further possible that the drugs contain antigen or antigen mimotopes as monomers, dimers, trimers or oligomers which are conjugated with the macromolecular carrier. Elevated immunogenicity is also produced here. Further, it is preferred that the monomeric, dimeric, trimeric or oligomeric antigens or antigen mimotopes are bound to the macromolecular carrier singly or multiply. This obtains an increase in immunogenicity.

With respect to the applicability of the antigenically acting substances in the inventive drugs, numerous possibilities are conceivable. One might mention for example antigenically acting substances associated with infectious diseases such as bacterial or viral infectious diseases.

Drugs in which the antigenically acting substance triggers an antitumoral effect are particularly preferred. Vaccinations and in particular the advantage of a possible oral vaccination are desirable for patients especially in the field of cancer therapy and cancer prophylaxis.

Some examples of the inventive oral vaccination will be explained hereinafter. The following show for explanation:

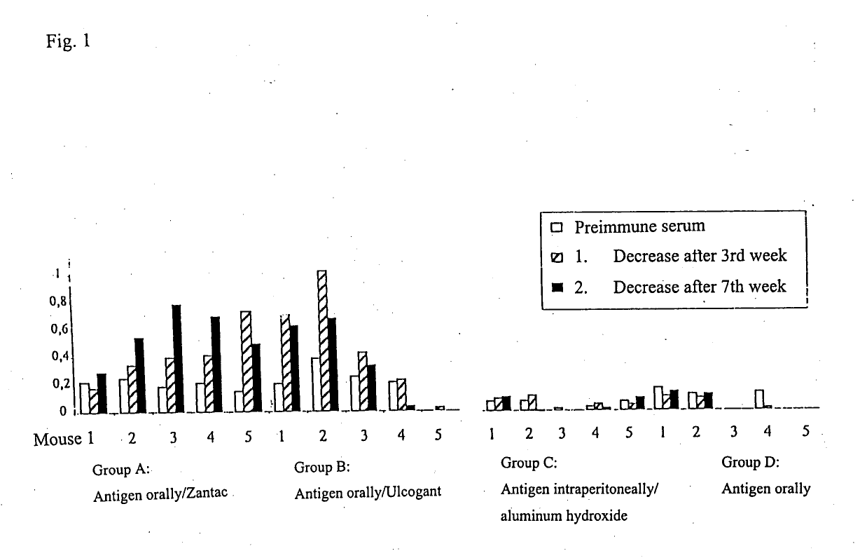

FIG. 1: A bar chart with the IgE increase in different mouse groups against antigenically used caviar extract (Example 1), measured by ELISA (scale: ODs), and

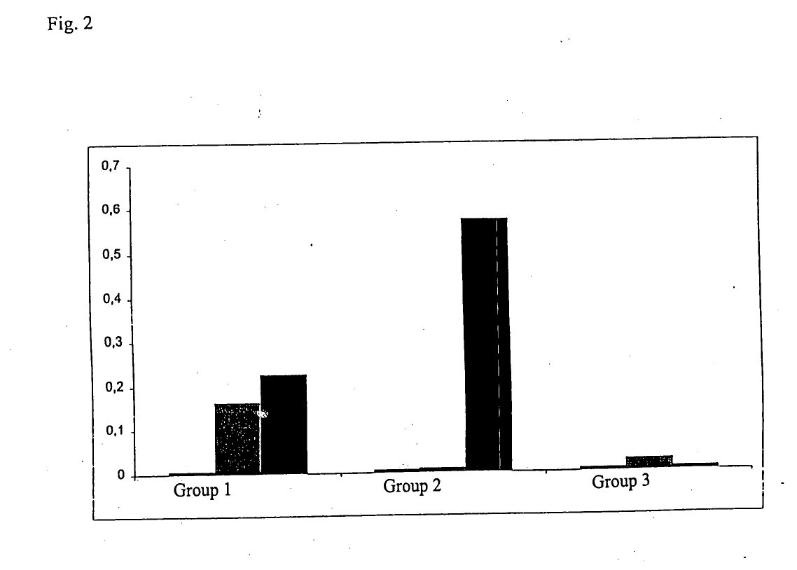

FIG. 2: A bar chart with the IgE increase in different mouse groups against antigenically used fish allergen, parvalbumin (Example 2), measured by ELISA (scale: ODs).

### EXAMPLE 1

Four groups (A to D) with five Balb/c mice each were treated in the following way:
Group A: Oral administration of Zantac (ULSAL®, active agent: 20 microg ranitidine per mouse, single dose per mouse one hour before feeding intramuscularly with antigen) and antigen (2 mg caviar protein extract per mouse),
Group B: Oral administration of a mixture of Ulcogant (active agent: 2 mg sucralfate per mouse) and antigen (2 mg caviar protein extract per mouse),
Group C: Comparative example with intraperitoneal administration of caviar antigen (2 mg) and aluminum hydroxide as an adjuvant,
Group D: Comparative example with oral administration of antigen (2 mg).

The antigen used was caviar extract. As a reference regarding the caviar extract used, the publication by Untersmayr E, Schöll I, Förster-Waldl E, Walter F. Riemer A, Boltz-Nitulescu G, Scheiner O, Jensen-Jarolim E. J Allergy and Immun. 2002; 109 (6): 1034-5 is cited. The antigen contained therein has a molecular weight of 118 kDa.

The obtained results are rendered in FIG. 1. FIG. 1 shows the IgE increases in the different mouse groups against the antigen, measured by ELISA (scale: ODs at 405/450 nm). Groups A and B were given the antigen in conjunction with a gastric acid reducing agent orally according to the invention, while for comparison purposes group C was immunized according to a standard scheme for IgE induction, and group D was given the antigen orally without gastric acid reducing agent.

The results according to the present invention make it clear that hypoacidity of the stomach (through antacid treatment) with simultaneous first-time intake of an antigen leads to an IgE induction specifically against said new antigen.

Common preparations that inhibit gastric acid formation (Zantac [ranitidine hydrochloride, G1 axoSmithKline]) or that bind and neutralize gastric acid (Ulcogant [aluminum sucrose hydrogen sulfate, Merck]) cause a lasting induction of IgE and also IgG antibodies against new food fed simultaneously or with a time delay, in this case caviar extract, but not against the daily diet of the mice, which they still tolerate. The achieved IgE values are also high in comparison with traditional allergization by the intraperitoneal route with Al(OH)₃ as an adjuvant.

### EXAMPLE 2

5 week old female Balb/c mice (n=8 per group) were treated by intragastric feedings each with 250 microg/mouse recombinant parvalbumin (fish allergen, see Swoboda et al., J. of Immunology 2002; 168: 4576-84). Group 1 (see FIG. 2, x-axis) was pretreated by Zantac (for quantity and active agent see Example 1) intramuscularly one hour before feeding, Group 2 with omeprazole intramuscularly (11.6 microg per mouse, Losec, AstraZeneca) two hours before feeding, Group three was given only parvalbumin fed without pretreatment.

The animals were treated on days 0 and 28. Sera were taken and tested by ELISA on day 0 (first column in each case), on day 28 (second column) and day 42 (third column). For this purpose ELISA plates were coated with recombinant parvalbumin. Mouse sera were diluted 1:10 and then incubated overnight. After washing, bound mouse IgE was detected by means of a peroxidase-labeled rat anti-mouse IgE antibody 1:1000 (PharMingen). The color reaction was caused by addition of substrate and is directly proportional to the quantity of specific IgE antibody. The color signal was read in an ELISA reader at 405/450 nm (FIG. 2, y-axis).

FIG. 2 shows the results obtained, the values shown in the diagram being the averages of the mouse groups (n=8). It can be concluded from the example of recombinant parvalbumin that pretreatments with antacids lead to fast IgE induction against new food antigens.

Due to the numerous literature reports indicating an antitumoral effect of IgE antibodies (Reali, E.; Greiner, J. W.; Corti, A.; Gould, H. J., Bottazzoli, F.; Paganelli, G.; Schlom, J.; Siccardi, A. G.; Cancer Res., 2001; 61 (14): 5517-22; Kershaw, M. H., Darcy, P. K.; Trapani, J. A.; MacGregor, D.; Smyth, M. J.; Oncol. Res., 1998, 10 (3), 133-42; Neuchrist, C.; Kornfehl, J.; Grasl, M.; Lassmann, H.; Kraft, D.; Ehrenberger, K.; Scheiner, O.; Int. Archs. Allergy Immunol., 1994, 104: 97-100; Nagy, E.; Berczi, I.; Sehon, A. H.; Cancer Immunol. Immunother. 1991; 34 (1): 63-9), a positive effect of vaccination by oral application of tumor mimotope antigens can also be expected due to the results of Examples 1 and 2.

1-10. (canceled)

11. A vaccine comprising an antigenically active substance and a gastric acid reducing substance.

12. The vaccine of claim 11, wherein the gastric acid reducing substance is present in an amount sufficient to increase the pH in the stomach to between pH 4 and pH 7.

13. The vaccine of claim 11, wherein the gastric acid reducing substance inhibits gastric acid formation or binds gastric acid.

14. The vaccine of claim 12, wherein the gastric acid reducing substance inhibits gastric acid formation or binds gastric acid.

15. The vaccine of claim 13, wherein the gastric acid reducing substance is selected from the group consisting of antacids, H₂-receptor antagonists and proton pump inhibitors.

16. The vaccine of claim 14, wherein the gastric acid reducing substance is selected from the group consisting of antacids, H₂-receptor antagonists and proton pump inhibitors.

17. The vaccine of claim 15, wherein the proton pump inhibitors are selected from the group consisting of omeprazole, lansoprazole, pantoprazole and rabeprazole.

18. The vaccine of claim 16, wherein the proton pump inhibitors are selected from the group consisting of omeprazole, lansoprazole, pantoprazole and rabeprazole.

19. The vaccine of claim 15, wherein the H₂ receptor antagonists are selected from the group consisting of cimetidine, ranitidine, omexetidine, famotidine, roxatidine and nizatidine.

20. The vaccine of claim 16, wherein the H₂ receptor antagonists are selected from the group consisting of cimetidine, ranitidine, omexetidine, famotidine, roxatidine and nizatidine.

21. The vaccine of claim 11, wherein the antigenically active substance is one or more natural antigens, synthetic antigens, antigen mimotopes, or a combination thereof.

22. The vaccine of claim 12, wherein the antigenically active substance is one or more natural antigens, synthetic antigens, antigen mimotopes, or a combination thereof.

23. The vaccine of claim 21, wherein the natural antigen or synthetic antigen and/or antigen mimotopes are coupled to a carrier.

24. The vaccine of claim 22, wherein the natural antigen or synthetic antigen and/or antigen mimotopes are conjugated to a carrier.

25. The vaccine of claim 23, wherein the natural antigen or synthetic antigen and/or antigen mimotopes are conjugated to a carrier.

26. The vaccine of claim 22, wherein the antigen mimotopes are conjugated to a carrier as a monomer, dimer, trimer, or oligomer.

27. The vaccine of claim 23, wherein the antigen mimotopes are conjugated to a carrier as a monomer, dimer, trimer, or oligomer.

28. The vaccine of claim 26, wherein single or multiple monomeric, dimeric, trimeric, or oligomeric antigen mimotopes are bound to the carrier.

29. The vaccine of claim 27, wherein single or multiple monomeric, dimeric, trimeric, or oligomeric antigen mimotopes are bound to the carrier.

30. The vaccine of claim 11, wherein the antigenically active substance is a tumor antigen.

31. The vaccine of claim 12, wherein the antigenically active substance is a tumor antigen.

32. A method of vaccination comprising orally administering to a subject in need thereof an effective amount of an antigenically active substance and an effective amount of a gastric acid reducing substance, wherein said method produces an immune response to the antigenically active substance.

33. A method of claim 32, wherein the gastric acid reducing substance is administered in an amount sufficient to increase the pH in the stomach to between pH 4 and pH 7.

34. The method of claim 32, wherein the antigenically active substance and gastric acid reducing substance are administered simultaneously.

35. The method of claim 32, wherein the antigenically active substance is administered after administration of the gastric acid reducing substance.

36. The method of claim 32, wherein the antigenically active substance and gastric acid reducing substance are released simultaneously in the stomach.

37. The method of claim 32, wherein the antigenically active substance is released in the stomach after release of the gastric acid reducing substance.

38. The method of claim 32, wherein the antigenically active substance is one or more natural antigens, synthetic antigens, antigen mimotopes, or a combination thereof.

39. The method of claim 38, wherein the natural antigen or synthetic antigen and/or antigen mimotopes are coupled to a carrier.

40. The method of claim 39, wherein the antigen mimotopes are conjugated to a carrier as a monomer, dimer, trimer, or oligomer.

41. The method of claim 32, wherein the antigenically active substance is a tumor antigen.

42. The method of claim 32, wherein the gastric acid reducing substance inhibits gastric acid formation or binds gastric acid.

43. The method of claim 32, wherein the gastric acid reducing substance is selected from the group consisting of antacids, H₂-receptor antagonists and proton pump inhibitors.

44. The method of claim 32, wherein the proton pump inhibitors are selected from the group consisting of omeprazole, lansoprazole, pantoprazole and rabeprazole.

45. The method of claim 15, wherein the H₂ receptor antagonists are selected from the group consisting of cimetidine, ranitidine, omexetidine, famotidine, roxatidine and nizatidine.

46. A method of vaccination comprising orally administering to a subject in need thereof the vaccine of claim 11, wherein said method produces an immune response to the antigenically active substance.

47. A method of vaccination comprising orally administering to a subject in need thereof the vaccine of claim 12, wherein said method produces an immune response to the antigenically active substance.

48. A method of vaccination comprising orally administering to a subject in need thereof the vaccine of claim 13, wherein said method produces an immune response to the antigenically active substance.

49. A method of vaccination comprising orally administering to a subject in need thereof the vaccine of claim 14, wherein said method produces an immune response to the antigenically active substance.

50. A method of vaccination comprising orally administering to a subject in need thereof the vaccine of claim 15, wherein said method produces an immune response to the antigenically active substance.

51. A method of vaccination comprising orally administering to a subject in need thereof the vaccine of claim 16, wherein said method produces an immune response to the antigenically active substance.

52. A method of vaccination comprising orally administering to a subject in need thereof the vaccine of claim 17, wherein said method produces an immune response to the antigenically active substance.

53. A method of vaccination comprising orally administering to a subject in need thereof the vaccine of claim 18, wherein said method produces an immune response to the antigenically active substance.

54. A method of vaccination comprising orally administering to a subject in need thereof the vaccine of claim 19, wherein said method produces an immune response to the antigenically active substance.

55. A method of vaccination comprising orally administering to a subject in need thereof the vaccine of claim 20, wherein said method produces an immune response to the antigenically active substance.

56. A method of vaccination comprising orally administering to a subject in need thereof the vaccine of claim 21, wherein said method produces an immune response to the antigenically active substance.

57. A method of vaccination comprising orally administering to a subject in need thereof the vaccine of claim 22, wherein said method produces an immune response to the antigenically active substance.

58. A method of vaccination comprising orally administering to a subject in need thereof the vaccine of claim 23, wherein said method produces an immune response to the antigenically active substance.

59. A method of vaccination comprising orally administering to a subject in need thereof the vaccine of claim 24, wherein said method produces an immune response to the antigenically active substance.

60. A method of vaccination comprising orally administering to a subject in need thereof the vaccine of claim 25, wherein said method produces an immune response to the antigenically active substance.

61. A method of vaccination comprising orally administering to a subject in need thereof the vaccine of claim 26, wherein said method produces an immune response to the antigenically active substance.

62. A method of vaccination comprising orally administering to a subject in need thereof the vaccine of claim 27, wherein said method produces an immune response to the antigenically active substance.

63. A method of vaccination comprising orally administering to a subject in need thereof the vaccine of claim 28, wherein said method produces an immune response to the antigenically active substance.

64. A method of vaccination comprising orally administering to a subject in need thereof the vaccine of claim 29, wherein said method produces an immune response to the antigenically active substance.

65. A method of vaccination comprising orally administering to a subject in need thereof the vaccine of claim 30, wherein said method produces an immune response to the antigenically active substance.

66. A method of vaccination comprising orally administering to a subject in need thereof the vaccine of claim 31, wherein said method produces an immune response to the antigenically active substance.


SEQUENCE LISTING (1 sequence)

SEQ ID NO: 1 - protein, 10 aa, Artificial Sequence
  Homo sapiens, mimotpe, selected by means of antibody (Herceptin)
QMWAPQWGPD